(19) United States
(12) Patent Application Publication
(10) Pub. No.: US 2007/0220684 A1
(43) Pub. Date: Sep. 27, 2007

(54) Methods, compositions, and kits for coloring hair

(76) Inventor: Saroja Narasimhan, Matawan, NJ (US)

Correspondence Address: Julie Blackburn;Revlon Consumer Products Corporation, Law Department, 237 Park Avenue, New York, NY, 10017, US

(21) Appl. No.: 11/385,096

(22) Filed: Mar. 21, 2006

(51) Int. Cl. A61K 8/00

(52) U.S. Cl. 8/405

(57) ABSTRACT

A water and oil emulsion developer composition for use in bleaching or oxidatively coloring hair comprising at least about 2-8% by weight of the total composition of oil phase and from about 1 to 5% by weight of the total composition of hydrogen peroxide wherein the ratio of the water to oil phase ranges from about 1.5 to 5 parts water phase to 1 part oil phase; and a method for oxidatively coloring hair with the composition.

### TECHNICAL FIELD

The invention is in the field of methods, compositions, and kits for coloring hair.

### BACKGROUND OF THE INVENTION

One common complaint among permanent, or oxidative, hair color users is that this type of hair color dries their hair. In fact, in some cases, oxidative hair color is not even recommended for consumers with unusually sensitive hair, or those that have damaged their hair due to excessive chemical processing. Yet, such consumers are not happy with semi-permanent, demi-permanent, or temporary hair color products that are milder to hair because the color washes out of the hair too readily. The consumer is then forced to repeat the coloration process at frequent intervals. Most consumers today lead very busy lives and do not have the time or inclination to spend excessive amounts of time on beauty rituals. In addition, there is a separate category of consumers that use chemical processes such as hair relaxing or permanent waving on their hair. Such processes can be somewhat damaging to hair. Often such consumers also wish to permanently color their hair. However, hair color professionals do not recommend oxidative hair color for such consumers because they believe that such overly sensitive hair may not withstand the further rigors of traditional oxidative hair color.

Accordingly, one significant need gap in the hair color category is to provide a hair color that permanently colors hair (also referred to as oxidative color), which is equivalent in mildness to semi-permanent, demi-permanent, or temporary hair color. Such a hair color would provide color equivalent in permanence to oxidative hair color (generally new hair growth requires re-coloring every four to six weeks) and the mildness associated with temporary, semi- or demi-permanent hair color processes.

In order to provide hair color compositions and processes that fulfill this need gap it is necessary to understand the oxidative hair color process and the ingredients used in the compositions. For example, in order to achieve permanent hair color, there must be lift as well as color deposit, The term “lift” refers to bleaching of the melanin found in hair fibers. When hair fibers are bleached, the color deposit on the hair fibers is more effective and the hair color produced more closely approximates the color promised on the package. For example, if the consumer is changing her hair color from brown to blonde, lift is critical. The brown melanin found in the hair fibers must be bleached so that the hair dye applied to the hair will provide the desired blonde shade. If this were not the case, the blonde hair dye mixture would simply deposit on the brown hair with the end result being a slightly lighter shade than the base brown hair color. In fact, this type of procedure is one method of coloring hair and is generally referred to as “tone on tone” hair color. In procedures where the hair color is not lifted but only color is deposited, the ultimate hair color can vary depending on the base hair color of the consumer prior to the procedure.

In oxidative hair color procedures, to achieve optimum penetration of oxidative hair dyes into the hair shaft, it is necessary to open the cuticles found on the hair surface so that the oxidative dyes can penetrate. This is traditionally accomplished by performing the hair color procedure at an alkaline pH sufficient to swell the hair shaft and open the cuticle. The aqueous oxidizing agent (hydrogen peroxide) composition, which is combined with the oxidative hair color composition immediately prior to use, has an alkaline pH sufficient to alkalize the hair color mixture applied to hair. It is known that the aqueous oxidizing agent (hydrogen peroxide) composition that is necessary in standard oxidative hair color processes can contribute to the hair becoming dry and brittle. Yet in order to achieve optimum lift it is essential that the procedure take place at a fairly high alkaline pH.

Hair color manufacturers are on an eternal quest to formulate oxidative hair color compositions and processes that will enable permanent coloring of hair but without the drawbacks often seen with the procedure. The hair color will provide bright, true, permanent color and be mild to hair, in fact as mild as temporary or demi-permanent color.

Accordingly, it is an object of the invention to provide a process and compositions for oxidatively coloring hair that provide optimal color deposit, lift and vibrancy yet are mild.

It is a further object of the invention to provide a method and compositions for oxidatively coloring hair with an aqueous oxidizing agent composition in the emulsion form wherein the oxidizing agent is concentrated in the water phase of the emulsion.

It is a further object of the invention to provide an improved process for the oxidative coloration of hair that provides color deposition, lift and vibrancy that is at least equivalent to standard oxidative procedures and compositions, yet is milder to the hair.

It is a further object of the invention to provide a method for oxidatively coloring hair with a developer composition having a considerable portion of conditioning oil and wherein the hydrogen peroxide concentration in the composition is considerably reduced when compared to standard compositions.

### SUMMARY OF THE INVENTION

The invention is directed to a method for oxidatively coloring hair comprising applying to the hair an oxidative hair color mixture prepared by combining at least one oxidative dye composition and at least one water and oil emulsion developer containing hydrogen peroxide from about 2 to 8% by weight of the total oil phase and 1 to 5% by weight of the total composition and wherein the ratio of water to oil phase ranges from about 1.5 to 5 parts water phase to 1 part oil phase.

### DETAILED DESCRIPTION

The invention is directed to a method for oxidatively coloring hair with a developer composition and an oxidative dye composition having certain specific ratios of ingredients.

I. The Developer Composition

The developer composition comprises a certain ratio of water phase, oil phase, and hydrogen peroxide.

A. Water

The developer composition comprises from about 1 to 80%, preferably from about 5 to 75%, more preferably from about 10-70% by weight of the total composition of water. In one preferred embodiment the developer composition comprises greater than about 75%, more preferably greater than about 77% by weight of the total composition of water phase.

B. Humectants

The developer composition may contain one or more humectants. If present, such humectants may range from about 0.01-25%, preferably about 0.05-15%, more preferably about 0.1-10% by weight of the total composition. Suitable humectants include mono-, di-, or polyhydric alcohols, and similar water soluble ingredients. Examples of suitable monohydric alcohols include ethanol, isopropanol, benzyl alcohol, butanol, pentanol, ethoxyethanol, and the like. Examples of dihydric, or polyhydric alcohols, as well as sugars and other types of humectants that may be used include glucose, fructose, mannose, mannitol, malitol, lactitol, inositol, and the like. Suitable glycols include propylene glycol, butylene glycol, ethylene glycol, polyethylene glycols having from 4 to 250 repeating ethylene glycol units, ethoxydiglycol, and the like.

C. Chelating Agents

The developer composition may contain one or more chelating agents which are capable of complexing with and inactivating metallic ions in order to prevent their adverse effects on the stability or effects of the composition. If present, suggested ranges are from about 0.0001-5%, preferably 0.0005-3%, more preferably 0.001-2% by weight of the total developer composition. Suitable chelating agents include EDTA and calcium, sodium, or potassium derivatives thereof, HEDTA, sodium citrate, TEA-EDTA, and so on.

D. pH Adjusters

It may also be desireable to add small amounts of acids or bases to adjust the pH of the developer composition to the desired pH range of greater than about 7.0 to 12.0. Suitable acids include hydrochloric acid, phosphoric acid, erythorbic acid, and the like. Suitable bases include sodium hydroxide, potassium hydroxide, disodium phosphate, and the like. Also suitable are primary secondary, or tertiary amines or derivative thereof such as aminomethyl propanol, monoethanolamine, and the like. Suggested ranges of pH adjusters are from about 0.00001-8%, preferably about 0.00005-6%, more preferably about 0.0001-5% by weight of the total composition.

E. Preservatives

The developer composition may also contain one or more preservatives. Suggested ranges are about 0.0001-8%, preferably 0.0005-7%, more preferably about 0.001-5% by weight of the total composition. Suitable preservatives include methyl, ethyl, and propyl paraben, hydantoins, and the like.

F. Oil Ingredients

The developer comprises from about 15 to 80%, preferably from about 20-75%, more preferably from about 25 to 65% by weight of the total developer composition of one or more oily ingredients. The term “oily ingredients” means ingredients that are soluble in the oil phase, or form part of the oil phase when the composition is in the emulsion form. The oily ingredients preferably have a conditioning benefit, e.g. will improve the condition of the hair whether used alone or in combination with other oil phase ingredients. Suitable oily ingredients include animal, vegetable, mineral, or synthetic oils, polymers, and so on. Preferably the composition comprises greater than about 25%, greater than 27% by weight of the total composition of oil phase.

Preferably, the one or more oils present may be hydrophobic in character. The term “hydrophobic” generally means that such oil has a Hildebrand solubility parameter (δ) measured in (cal/cm ³)^{1/2} (the square root of calories per cubic centimeter), ranging from about 5 to 12, preferably from about 7 to 9. The term “hydrophobic” means that the oil is lipophilic in character. The hydrophobic oil is preferably a liquid at room temperature (250° C.).

The term “solubility parameter” when used in accordance with this invention means the Hildebrand solubility parameter (δ) which is calculated according to the formula:
δ=(ΔEv/V)^{1/2}
wherein ΔEv=heat of vaporization of the particular ingredient, and V=molecular weight/density of the ingredient.

The Hildebrand solubility parameters (δ) are generally available by referring to standard chemistry textbooks or similar reference manuals. The Journal of the Society of Cosmetic Chemistry, Volume 36, pages 3199-333, and Cosmetics and Toiletries, Vol. 103, October 1988, pages 47-69, both of which are hereby incorporated by reference in their entirety, list the Hildebrand solubility parameter (δ) values for a wide variety of cosmetic ingredients and how the solubility parameter is calculated.

Oils which have solubility parameters which are significantly less than 5 are extremely hydrophobic, and while such oils may promote preferential absorption of the active ingredients into the hair shaft by causing the water soluble actives to “repel” the oil and be preferentially pushed into the hair shaft, they are difficult to disperse in the developer composition because of their extreme lipophilic character. Oils which have Hildebrand solubility parameters which are significantly greater than 12 are too hydrophilic and will more readily form part of the water phase of the emulsion developer composition. Accordingly, such oils will not induce preferential absorption of the water soluble active ingredients into the hair shaft as such active ingredients are compatible with the hydrophilic ingredients present in the composition.

1. Vegetable Oils

Suitable vegetable oils include those derived from plants such as leaves, stems, bark, flowers, fruit, etc. Examples include oils from coconut, grape seed, grape, apple seed, coconut oil, mineral oil, isopropyl myristate, linseed oil, octyl palmitate, safflower, corn, poppy seed, corn, currant, peanut, almond, cotton seed, birch, pine seed, pine nut, and so on. Preferred is coconut oil, mineral oil, or mixtures thereof.

2. Hydrocarbons

Suitable hydrocarbons include mineral oil, squalane, squalene, polybutene, polyisobutene, polydecene, and hydrogenated derivatives thereof.

3. Cationic Silicones

As used herein, the term “cationic silicone” means any silicone polymer or oligomer having a silicon backbone, including polysiloxanes, having a positive charge on the silicone structure itself. Cationic silicones that may be used in the developer compositions of the invention include those corresponding to the following formula, where the ratio of D to T units, if present are greater than about 80 D units to 1 T unit:
(R)ₐG₃₋ₐ-Si—(—OSiG₂)ₙ-(—OSiG_{b}(R₁)2-6b)ₘ—O—SiG₃₋ₐ(R₁)ₐ
in which G is selected from the group consisting of H, phenyl, OH, C₁₋₁₀ alkyl, and is preferably CH₃; and a is 0 or an integer from 1 to 3, and is preferably 0; b is 0 or 1, preferably 1; the sum n+m is a number from 1 to 2,000 and is preferably 50 to 150; n is a number from 0 to 2000, and is preferably 50 to 150; and m is an integer from 1 to 2000, and is preferably 1 to 10; R is a C₁₋₁₀ alkyl and R₁ is a monovalent radical of the formula C_{q}H_{2q}L in which q is an integer from 2 to 8 and L is selected from the groups: 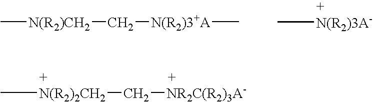
in which R₂ is selected from the group consisting of H, phenyl, benzyl, a saturated hydrocarbon radical, and is preferably an alkyl radical containing 1-20 carbon atoms; and A- is a halide, methylsulfate, or tosylate ion.

4. Lipophilic Polymers

A variety of lipophilic polymers are suitable for use in the developer composition, including copolymers of vinylpyrrolidone, polymers of dimethyldiallyl ammonium chloride, acrylic or methacrylic polymers, quaternary ammonium polymers, and the like.

(a). Copolymers of Vinylpyrrolidone

Copolymers of vinylpyrrolidone having monomer units of the formula: 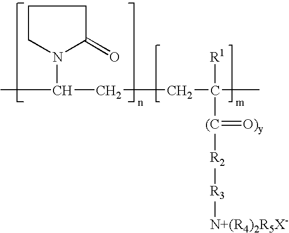
wherein R¹ is hydrogen or methyl, preferably methyl;
y is 0 or 1, preferably 1
R² is O or NH, preferably NH;
R³ is CₓH₂ₓ where x is 2 to 18, or —H₂—CHOH—CH₂, preferably CₓH₂ₓ
where x is 2;
R⁴ is methyl, ethyl, phenyl, or C₁₋₄ substituted phenyl, preferably methyl; and
R⁵ is methyl or ethyl, preferably methyl.

(b). Polymers of Dimethyldiallylammonium Chloride

Homopolymers of dimethyldiallylammonium chloride or copolymers of dimethyldiallylammonium chloride and acrylamide are also suitable. Such compounds are sold under the tradename MERQUAT by Calgon.

(c). Acrylic or Methacrylic Acid Polymers

Homopolymers or copolymers derived from acrylic or methacrylic acid, selected from monomer units acrylamide, methylacrylamide, diacetone-acrylamide, acrylamide or methacrylamide substituted on the nitrogen by lower alkyl, alkyl esters of acrylic acid and methacrylic acid, vinylpyrrolidone, or vinyl esters are suitable for use.

(d). Nonionic Silicones

Also suitable as conditioning agents are one or more silicones. Suitable silicone hair conditioning agents include volatile or nonvolatile nonionic silicone fluids, silicone resins, and silicone semi-solids or solids.

Volatile silicones are linear or cyclic silicones having a measureable vapor pressure, which is defined as a vapor pressure of at least 2 mm. of mercury at 20° C. Examples of volatile silicones are cyclic silicones having the general formula: 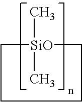
where n =3-7.

Also, linear volatile silicones that may be used in the compositions of the invention have the general formula:
(CH₃)₃Si—O—[Si(CH₃)₂—O]ₙ—Si(CH₃)₃
where n=0-7, preferably 0-5.

Also suitable are nonvolatile silicone fluids including polyalkyl siloxanes, polyaryl siloxanes, polyalkylaryl siloxanes, polyether siloxane copolymers, amine-functional silicones, and mixtures thereof. Such silicones have the following general formula: 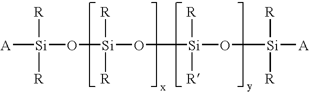
wherein R and R′ are each independently alkyl, aryl, or an alkyl substituted with one or more amino groups, and x and y are each independently 0-100,000, with the proviso that x+y equals at least one, and A is siloxy endcap unit. Preferred is where A is methyl, R is methyl, and R′ is an alkyl substituted with at least two amino groups, most preferably an amine-functional silicone having the formula: 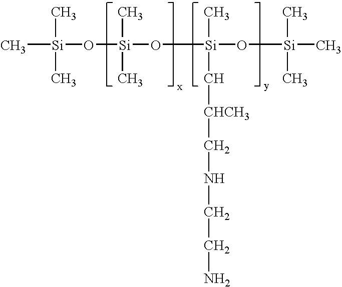
which is known by the CTFA name trimethylsilylamodimethicone.

Another type of silicone conditioning agent is a silicone polymer having the following general formula: 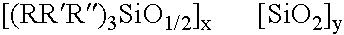
wherein R, R′ and R″ are each independently a C₁₋₁₀ straight or branched chain alkyl or phenyl, and x and y are such that the ratio of (RR′R″)₃SiO_{1/2} units to SiO₂ units is 0.5 to 1 to 1.5 to 1.

Preferably R, R′ and R″ are a C₁₋₆ alkyl, and more preferably are methyl and x and y are such that the ratio of (CH₃)₃SiO_{1/2} units to SiO₂ units is 0.75 to 1. Most preferred is this trimethylsiloxy silicate containing 2.4 to 2.9 weight percent hydroxyl groups, which is formed by the reaction of the sodium salt of silicic acid, chlorotrimethylsilane, and isopropyl alcohol. The manufacture of trimethylsiloxy silicate is set forth in U.S. Pat. Nos. 2,676,182; 3,541,205; and 3,836,437, all of which are hereby incorporated by reference. Trimethylsiloxysilicate as described is available from Dow Corning Corporation under the tradename Dow Corning 749 Fluid, which is a blend of about 40-60% volatile silicone and 40-60% trimethylsiloxy silicate (trimethylated silica). The fluid has a viscosity of 200-700 centipoise at 250° C., a specific gravity of 1.00 to 1.10 at 25° C., and a refractive index of 1.40-1.41.

G. Thickeners

The developer composition may contain one or more thickeners that assist in maintaining an increased viscosity of the final composition resulting from mixture of the hair dye and the developer compositions. The amount of thickening agent if present is about 0.001-5%, preferably about 0.005-4%, more preferably about 0.005-3% by weight of the total composition.

A variety of thickening agents are suitable including low melting point waxes, carboxyvinyl polymers, and the like. Also suitable are a variety of water soluble anionic thickening polymers such as those disclosed in U.S. Pat. No. 4,240,450, which is hereby incorporated by reference. Suggested ranges of such polymers are about 0.01-5%, preferably 0.05-4%, more preferably 0.1-3% by weight of the total developer composition. Examples of such anionic polymers are copolymers of vinyl acetate and crotonic acid, graft copolymers of vinyl esters or acrylic or methacrylic acid esters, cross-linked graft copolymers resulting from the polymerization of at least one monomer of the ionic type, at least one monomer of the nonionic type, polyethylene glycol, and a crosslinking agent, and the like. Preferred are acrylate copolymers such as steareth-10 allyl ether acrylate copolymer.

H. Nonionic Surfactants

The developer composition may contain one or more nonionic surfactants. Suggested ranges of nonionic surfactant, if present are about 0.01-10%, preferably about 0.05-8%, more preferably about 0.1-7% by weight of the total composition. Suitable nonionic surfactants include alkoxylated alcohols or ethers, alkoxylated carboxylic acids. sorbitan derivatives, and the like.

1. Alkoxylated Alcohols

Suitable alkoxylated alcohols, or ethers, are formed by the reaction of an alcohol with an alkylene oxide, usually ethylene or propylene oxide. Preferably the alcohol is a fatty alcohol having 6 to 30 carbon atoms, and a straight or branched, saturated or unsaturated carbon chain. Examples of such ingredients include steareth 2-30, which is formed by the reaction of stearyl alcohol and ethylene oxide where the number of repeating ethylene oxide units is 2 to 30; Oleth 2-30 which is formed by the reaction of oleyl alcohol and ethylene oxide where the number of repeating ethylene oxide units is 2 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on. Particularly preferred are Steareth-21, which is the reaction product of a mixture of stearyl alcohol with ethylene oxide, and the number of repeating ethylene oxide units in the molecule is 21, and Oleth-20 which is the reaction product of oleyl alcohol and ethylene oxide wherein the number of repeating ethylene oxide units in the molecule is 20.

2. Alkoxylated Carboxylic Acids

Also suitable as the nonionic surfactant are alkyoxylated carboxylic acids, which are formed by the reaction of a carboxylic acid with an alkylene oxide or with a polymeric ether. The resulting products have the general formula: 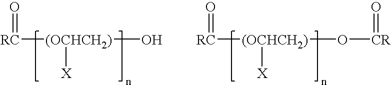
where RCO is the carboxylic ester radical, X is hydrogen or lower alkyl, and n is the number of polymerized alkoxy groups. In the case of the diesters, the two RCO— groups do not need to be identical. Preferably, R is a C₆₋₃₀ straight or branched chain, saturated or unsaturated alkyl, and n is from 1-100.

3. Alkoxylated Sorbitans

Also suitable are various types of alkoxylated sorbitan and alkoxylated sorbitan derivatives. For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate. sorbitan stearate, and so on.

I. Anionic Surfactants

If desired the developer composition may contain one or more anionic surfactants. Preferred ranges of anionic surfactant, if present, are from about 0.1-25%, preferably 0.5-20%, more preferably 1-15% by weight of the total composition. Suitable anionic surfactants include alkyl and alkyl ether sulfates generally having the formula ROSO₃M and RO(C₂H₄O)ₓSO₃M wherein R is alkyl or alkenyl of from about 10 to 20 carbon atoms, x is 1 to about 10 and M is a water soluble cation such as ammonium, sodium, potassium, or triethanolamine cation.

Another type of anionic surfactant which may be used in the compositions of the invention are water soluble salts of organic, sulfuric acid reaction products of the general formula:
R₁—SO₃—M
wherein R₁ is chosen from the group consisting of a straight or branched chain, saturated aliphatic hydrocarbon radical having from about 8 to about 24 carbon atoms, preferably 12 to about 18 carbon atoms; and M is a cation. Examples of such anionic surfactants are salts of organic sulfuric acid reaction products of hydrocarbons such as n-paraffins having 8 to 24 carbon atoms, and a sulfonating agent, such as sulfur trioxide.

Also suitable as anionic surfactants are reaction products of fatty acids esterified with isethionic acid and neutralized with sodium hydroxide. The fatty acids may be derived from coconut oil, for example.

In addition, succinates and succinimates are suitable anionic surfactants. This class includes compounds such as disodium N-octadecylsulfosuccinate; tetrasodium N-(1,2-dicarboxyethyl)-N-octadecylsulfosuccinate; and esters of sodium sulfosuccinic acid e.g. the dihexyl ester of sodium sulfosuccinic acid, the dioctyl ester of sodium sulfosuccinic acid, and the like.

Other suitable anionic surfactants include olefin sulfonates having about 12 to 24 carbon atoms. The term “olefin sulfonate” means a compound that can be produced by sulfonation of an alpha olefin by means of uncomplexed sulfur trioxide, followed by neutralization of the acid reaction mixture in conditions such that any sultones which have been formed in the reaction are hydrolyzed to give the corresponding hydroxy-alkanesulfonates. The alpha-olefin from which the olefin sulfonate is derived is a mono-olefin having about 12 to 24 carbon atoms, preferably about 14 to 16 carbon atoms.

Other classes of suitable anionic organic surfactants are the beta-alkoxy alkane sulfonates or water soluble soaps thereof such as the salts of C₁₀₋₂₀ fatty acids, for example coconut and tallow based soaps. Preferred salts are ammonium, potassium, and sodium salts.

Still another class of anionic surfactants include N-acyl amino acid surfactants and salts thereof (alkali, alkaline earth, and ammonium salts) having the formula: 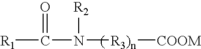
wherein R₁ is a C₈₋₂₄ alkyl or alkenyl radical, preferably C₁₀₋₁₈; R₂ is H, C₁₋₄ alkyl, phenyl, or —CH₂COOM; R₃ is CX₂— or C₁₋₂ alkoxy, wherein each X independently is H or a C₁₋₆ alkyl or alkylester, n is from 1 to 4, and M is H or a salt forming cation as described above. Examples of such surfactants are the N-acyl sarcosinates, including lauroyl sarcosinate, myristoyl sarcosinate, cocoyl sarcosinate, and olcoyl sarcosinate, preferably in sodium or potassium forms.

J. Amphoteric or Zwitterionic Surfactants

Also suitable are amphoteric and zwitterionic surfactants. Examples of amphoteric surfactants that can be used in the compositions of the invention are generally described as derivatives of aliphatic secondary or teniary amines wherein one aliphatic radical is a straight or branched chain alkyl of 8 to 18 carbon atoms and the other aliphatic radical contains an anionic group such as carboxy, sulfonate, sulfate, phosphate, or phosphonate.

K. Fatty Alcohols

The developer composition may also contain one or more fatty alcohols of the general formula R—OH wherein R is a C₆₋₄₀ straight or branched chain, saturated or unsaturated alkyl. Preferred alcohols include cetearyl alcohol, stearyl alcohol, cetyl alcohol, and the like. If present the fatty alcohol may range from about 0.1-10%, preferably from about 0.5-8%, more preferably from about 1-7% by weight of the total composition.

The developer composition may contain one or more additional ingredients including but not limited to botanicals, antioxidants, polymers, pigments, and the like. The developer composition of the invention has a concentration of hydrogen peroxide ranging from about 2 to 8% by weight of the total oil phase and 1 to 5% by weight of the total composition and wherein the ratio of water to oil phase ranges from about 1.5 to 5 parts water phase to 1 part oil phase. In the tables below, the concentration of hydrogen peroxide in developer compositions ranging from 5 to 30 Volume are set forth, both by weight of the total composition and by weight of the oil phase of the composition. The developer compositions used in the method of the invention include those having 5 to 15 Volumes of hydrogen peroxide.

| Hydrogen Peroxide | Hydrogen Peroxide | Hydrogen Peroxide |
|---|---|---|
| Composition Volume | Concentration % | Concentration % |
| Concentration-Total | by Weight | by Weight |
| Composition | Total Composition | Water Phase |
| 5 | 1.5 | 2.3 |
| 10 | 3 | 4.6 |
| 15 | 4.5 | 6.9 |
| 20 | 6 | 9.2 |
| 25 | 7.5 | 11.5 |
| 30 | 9 | 13.8 |

The table below illustrates the ratio of water phase and oil phase in the developer compositions used in the method of the invention compared to standard developer compositions.

In the developer compositions used in the method of the invention, the ratio of water phase to oil phase is from about 1.5 to 5 parts water phase to 1 part oil phase, preferably from about 2 to 4 parts water phase to 1 part oil phase.

| Developer | Percent | Percent | Ratio Water |
|---|---|---|---|
| Volume | Water | Oil | Phase to |
| Concentration | Phase | Phase | Oil Phase |
| 10 Volume | 68 | 32 | 2.12 |
| 15 Volume | 68 | 32 | 2.12 |
| 20 Volume | 91.64 | 8.36 | 10.96 |
| (Colorsilk Hair | | | |
| Color) | | | |
| 30 Volume | 94.88 | 5.12 | 18.53 |
| (Colorsilk Hair | | | |
| Color) | | | |

L. Microemulsion or Liquid Crystalline Form

If desired the developer composition may be in the form of a microemulsion or liquid crystalline format, as set forth in U.S. Pat. Nos. 6,315,989 and 6,238,653 both of which are hereby incorporated by reference in their entirety. In the case where the developer composition is in the microemulsion form, the dispersed water droplets have particle sizes ranging from 100 to 1500 Angstroms. In the case where the developer composition is in the form of a liquid crystalline composition, the composition contains water, oil, and an appropriate amount of an amphiphilic surface active material that is operable to form liquid crystals.

Preferably, the developer composition is in the form of a water-in-oil or oil-in-water microemulsion having dispersed water microdroplets in the oil phase. Without being bound by this explanation, it is believed that while the concentration of hydrogen peroxide is lower in the total composition, it is more concentrated in the aqueous phase, particularly when the developer is in the form of a microemulsion. Accordingly, the developer is more effective. The relatively high concentration of oil phase forces the hydrogen peroxide into the water phase. When applied to the hair after mixing with the oxidative dye composition, the hydrogen peroxide containing water phase is “pushed into” the hair as it repels the oily phase. The oily phase remains on the hair surface after the aqueous phase has penetrated and forms a coating that conditions the hair. In general, when the developer composition is in this form it is more efficacious in activating the oxidative dye composition, thus a significantly lower concentration of hydrogen peroxide is necessary.

In the method of the invention the developer composition is combined with the oxidative dye composition and applied to the hair in order to color the hair.

II. The Oxidative Dye Composition

In general, the oxidative dye compositions used in the method are aqueous based and comprise about 0-20%, preferably about 0.001-10%, more preferably about 0.01-8% by weight of the total oxidative dye composition of dyestuff components and about 0.0001-99.9%, preferably about 0.001-98%, more preferably about 0.001-90% by weight of the total composition of water base.

A. Dyestuff Components

Dyestuff components include primary intermediates and, optionally, couplers for the formation of oxidation dyes.

Suggested ranges of primary intermediates present in the oxidative dye composition range from about 0-6%, preferably about 0.0001-5.5%, more preferably about 0.001-5% by weight of the total composition. Such primary intermediates are well known for use in hair color, and include ortho or para substituted aminophenols or phenylenediamines, such as para-phenylenediamines of the formula: 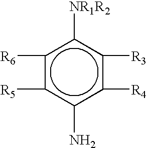
wherein R₁ and R₂ are each independently hydrogen, C₁₋₆ alkyl, or C₁₋₆ alkyl substituted with one or more hydroxy, methoxy, methylsulphonylamino, aminocarbonyl, furfuryl, unsubstituted phenyl, or amino substituted phenyl groups; R₃, R₄, R₅, and R₆ are each independently hydrogen, C₁₋₆ alkyl, C₁₋₆ alkoxy, halogen, or C₁₋₆ alkyl substituted with one or more hydroxy or amino groups.

Specific examples of suitable primary intermediates include para-phenylenediamine, 2-methyl-1,4-diaminobenzene, 2,6-dimethyl-1,4-diaminobenzene, 2,5-dimethyl-1,4-diamninobenzene, 2,3-dimethyl-1,4-diaminobenzene, 2-chloro-1,4-diaminobenzene, 2-methoxy-1,4-diaminobenzene,1-phenylamino-4-aminobenzene, 1-dimethylamino-4-aminobenzene, 1-diethylamino-4-aminobenzene, 1-bis(beta-hydroxyethyl)amino-4-aminobenzene, 1-methoxyethylamino-4-aminobenzene, 2-hydroxymethyl-1,4-diaminobenzene, 2-hydroxyethyl-1,4-diaminobenzene, 2-isopropyl-1,4-diaminobenzene, 1-hydroxypropylamino-4-aminobenzene, 2,6-dimethyl-3-methoxy-1,4-diaminobenzene, 1-amino-4-hydroxybenzene, and derivatives thereof, and acid or basic salts thereof.

Preferred primary intermediates are p-phenylenediamine, p-aminophenol, o-aminophenol, N,N-bis(2-hydroxyethyl)-p-phenylenediamine, 2,5-diaminotoluene, their salts and mixtures thereof.

Suitable color couplers, if present, range from about 0.0001-10%, more preferably about 0.0005-8%, most preferably about 0.001-7% by weight of the total oxidative dye composition. Such color couplers include, for example, those having the general formula: 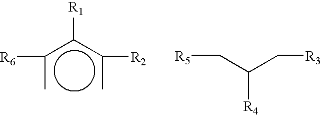
wherein R₁ is unsubstituted hydroxy or amino, or hydroxy or amino substituted with one or more C₁₋₆ hydroxyalkyl groups. R₃ and R₅ are each independently hydrogen, hydroxy, amino, or amino substituted with C₁₋₆ alkyl, C₁₋₆ alkoxy, or C₁₋₆ hydroxyalkyl group; and R₂, R₄, and R₆ are each independently hydrogen, C₁₋₆ alkoxy, C₁₋₆ hydroxyalkyl, or C₁₋₆ alkyl, or R₃ and R₄ together may form a metliylenedioxy or ethylenedioxy group. Examples of such compounds include meta-derivatives such as phenols, catechol, meta-aminophenols, meta-phenylenediamines, and the like, which may be unsubstituted, or substituted on the amino group or benzene ring with alkyl, hydroxyalkyl, alkylamino groups, and the like. Suitable couplers include m-aminophenol, 2,4-diaminotoluene, 4-amino, 2-hydroxytoluene, phenyl methyl pyrazolone, 3,4-methylenedioxyphenol, 3,4-methylenedioxy-1-[(beta-hydroxyethyl)amino]benzene, 1-methoxy-2-amino-4-[(beta-hydroxyethyl)amino]benzene,1-hydroxy-3-(dimethylamino)benzene, 6-methyl-1-hydroxy-3[(beta-hydroxyethyl)amino]benzene, 2,4-dichloro-1-hydroxy-3-aminobenzene, 1-hydroxy-3-(diethylamino)benzene, 1-hydroxy-2-methyl-3-aminobenzene, 2-chloro-6-methyl-1-hydroxy-3-aminobenzene, 1,3-diaminobenzene, 6-methoxy-1,3-diaminobenzene, 6-hydroxyethoxy-1,3-diaminobenzene, 6-methoxy-5-ethyl-1,3-diaminobenzene, 6-ethoxy-1,3-diaminobenzene, 1-bis(beta-hydroxyethyl)amino-3-aminobenzene, 2-methyl-1,3-diaminobenzene, 6-methoxy-1-amino-3-[(beta-hydroxyethyl)amino]-benzene, 6-(beta-aminoethoxy)-1,3-diaminobenzene, 6-(beta-hydroxyethoxy)-1-amino-3-(methylamino)benzene, 6-carboxymethoxy-1,3-diaminobenzene. 6-ethoxy-1-bis(beta-hydroxyethyl)amino-3-aminobenzene, 6-hydroxyethyl-1,3-diaminobenzene, 1-hydroxy-2-isopropyl-5-methylbenzene, 1,3-dihydroxybenzene, 2-chloro-1,3-dihydroxybenzene, 2-methyl-1,3-dihydroxybenzene, 4-chloro-1,3-dihydroxybenzene, 5,6-dichloro-2-methyl-1,3-dihydroxybenzene, 1-hydroxy-3-amino-benzene, 1-hydroxy-3-(carbamoylmethylamino)benzene, 6-hydroxybenzomorpholine, 4-methyl-2,6-dihydroxypyridine, 2,6-dihydroxypyridine, 2,6-diaminopyridine, 6-aminobenzomorpholine, 1-phenyl-3-methyl-5-pyrazolone, 1-hydroxynaphthalene, 1,7-dihydroxynaphthalene, 1,5-dihydroxynaphthalene, 5-amino-2-methyl phenol, 4-hydroxyindole, 4-hydroxyindoline, 6-hydroxyindole, 6-hydroxyindoline, 2,4-diamioniphenoxyethanol, and mixtures thereof.

Preferred couplers include resorcinol, 1-naphthol, 2-methylresorcinol, 4-amino-2-hydroxy toluene, m-aminophenol, 2,4-diaminophenoxyethanol, phenyl methyl pyrazolone, their salts, or mixtures.

In the haircolor industry, haircolor is classified into one of ten levels as follows:

```
  1 = very black
  2 = bright black
  3 = very dark brown
  4 = dark brown
  5 = medium brown
  6 = light brown
  7 = dark blonde
  8 = medium blonde
  9 = light blonde
10 = high lift blonde
```

Set forth in the table below is a non-limiting example of the primary intennediates and the color couplers that may be used in various shades of hair color. Other primary intermediates and couplers may be used in addition to, or in lieu oft those set forth in the Table and nothing herein shall be construed to limit the invention to only those primary intermediates and couplers set forth.

| Level 1—Very Black | | Level 2—Bright Black | |
|---|---|---|---|
| Primary | | Primary | |
| Intermediates | Couplers | Intermediates | Couplers |
| p-phenylenediamine | m-aminophenol | p-phenylenediamine | resorcinol |
| p-phenylenediamine | resorcinol | 2-chloro-P- | |
| sulfate | | phenylene- | |
| | | diamine sulfate | |
| 2-chloro-phenylene | 4-amino-2-hydroxy | o-aminophenol | |
| diamine sulfate | toluene | | |
| p-aminophenol | 4-chlororesorcinol | | |
| o-aminophenol | m-aminophenol | | |
| | HCL | | |
| | 2,4-diaminophenoxy | | |
| | ethanol | | |
| | m-phenylenediamine | | |
| | sulfate | | |
| Level 3—Very Dark Brown | | Level 4—Dark Brown | |
| Primary | | Primary | |
| Intermediates | Couplers | Intermediates | Couplers |
| p-phenylenediamine | resorcinol | p-phenylenediamine | resorcinol |
| N,N-bis(2-hydroxy- | 1-naphthol | N,N-bis(2- | 1-naphthol |
| ethyl)-P- | | hydroxyethyl)-P- | |
| phenylene- | | phenylene diamine | |
| diamine sulfate | | sulfate | |
| | m-aminophenol | p-aminophenol | m-aminophenol |
| | | | phenyl methyl |
| | | | pyrazolone |
| | | o-aminophenol | 4-amino-2- |
| | | | hydroxytoluene |
| Level 5—Medium Brown | | Level 6—Light Brown | |
| Primary | | Primary | |
| Intermediates | Couplers | Intermediates | Couplers |
| p-phenylenediamine | resorcinol | p-phenylenediamine | resorcinol |
| N,N-bis(2-hydroxy- | 1-naphthol | N,N-bis(2-hydroxy- | 1-naphthol |
| ethyl)-P-phenylene | | ethyl)-P-phenylene | |
| diamine sulfate | | diamine sulfate | |
| p-aminophenol | m-aminophenol | p-aminophenol | m-aminophenol |
| o-aminophenol | phenyl methyl | | phenyl methyl |
| | pyrazolone | | pyrazolone |
| | 2-methylresorcinol | | 4-amino-2-hydroxy |
| | | | toluene |
| | 4-amino-2-hydrox- | | 2-methylresorcinol |
| | toluene | | |
| Level 7—Dark Blonde | | Level 8—Medium Blonde | |
| Primary | | Primary | |
| Intermediates | Couplers | Intermediates | Couplers |
| p-phenylenediamine | resorcinol | p-phenylenediamine | resorcinol |
| N,N-bis(2-hydroxy- | 1-naphthol | N,N-bis(2- | 1-naphthol |
| ethyl)-P-phenylene | | hydroxyethyl)-P- | |
| diamine sulfate | | phenylenediamine | |
| | | sulfate | |
| p-aminophenol | phenyl methyl | p-aminophenol | m-aminophenol |
| | pyrazolone | | |
| o-aminophenol | | | phenyl methyl |
| | | | pyrazolone |
| | | | 4-amino-2- |
| | | | hydroxytoluene |
| Level 9—Light Blonde | | Level 10—High Lift Blonde | |
| Primary | | Primary | |
| Intermediates | Couplers | Intermediates | Couplers |
| p-phenylenediamine | resorcinol | p-phenylenediamine | resorcinol |
| N,N-bis(2- | 4-amino-2-hydroxy | N,N-bis(2-hydroxy- | 1-naphthol |
| hydroxyethyl)-P- | toluene | ethyl)-P-phenylene- | |
| phenylenediamine | | diamine sulfate | |
| sulfate | | | |
| p-aminophenol | phenyl methyl | | phenyl methyl |
| | pyrazolone | | pyrazolone |
| o-aminophenol | 2-methylresorcinol | | 2-methylresorcinol |
| | 1-naphthol | | |

B. Alkalizing Agent

The oxidative dye composition may also contain one or more alkalizing agents preferably in a range of about 0.1-5% based on the total weight of the oxidative dye composition. The term “alkalizing agent” means an ingredient that is capable of imparting alkalinity (e.g. a pH of greater than 7) to the dye mixture. Suitable alkalizing agents include ammonium hydroxide, metal hydroxides, alkanolamines, sodium silicate. metal carbonates, sodium metasilicate, and mixtures thereof. Suitable metal hydroxides and carbonates include alkali metal and alkaline earth metal hydroxides or carbonates. Examples of such metal hydroxides include sodium, potassium, lithium, calcium, magnesium and so on. A particularly preferred alkaline earth metal hydroxide is sodium hydroxide. Suitable alkanolamines include mono-, di-, and trialkanolamines such as monoethanolamine (MEA), diethanolamine (DEA), triethanolamine (TEA), 2-aminobutanol, aminoethyl propanediol, aminomethyl propanediol, bis-hydroxyethyl tromethamine, diethanolamine, diethyl ethanolamine, diisopropanolamine, dimethylamino methylpropanol, dimethyl MEA, isopropanolamine, methylethanolamine, mixed isopropanolamines, triisopropanolamine, tromethamine, and mixtures thereof. A particularly preferred alkanolamine is MEA.

The alkalizing agent present in the hair dye mixture may react with other ingredients in the mixture in situ, such as fatty acids, proteins or hydrolyzed proteins, and the like. Depending on the amount of alkalizing agent present and the presence or absence of ingredients that will react with the alkalizing agent, it is possible that the alkalizing agent may be completely reacted in situ, partially reacted in situ, or not reacted at all if there are no other ingredients in the composition that will react with the alkalizing agent. Most preferred is where the oxidative dye composition comprises mixtures of alkalizing agents, in particular, ammonium hydroxide in combination with a second alkalizing agent such as an alkanolamine.

Other Ingredients

1. Fatty Acids

The composition may contain one or more fatty acids, and if so suggested ranges are about 0.001-15%, preferably 0.005-10%, most preferably 0.01-8% by weight of the total composition. If fatty acids are present they will react with the alkalizing agent to form soap in situ which provides a more shampoo-like character to the aqueous hair color composition once it is applied to hair. Such fatty acids are of the general formula RCOOH wherein R is a straight or branched chain, saturated or unsaturated C₆₋₃₀ alkyl. Examples of suitable fatty acids include oleic acid, stearic acid, myristic acid, linoleic acid, and so on. Particularly preferred is oleic acid.

2. Conditioners

The oxidative dye composition may comprise one or more conditioners that exert a conditioning effect on hair. Conditioners mentioned above with respect to the developer composition are suitable, in the percentage ranges suggested.

3. Surfactants

Surfactants may also be used in the oxidative dye compositions of the invention. Suitable surfactants include nonmonic, anionic, amphoteric, zwitterionic, and the like as set forth with respect to the developer composition and in the same general ranges.

4. Thickening Agents

Preferably the oxidative dye composition contains one or more thickening agents that increase the viscosity of the composition such that when it is applied to hair it doesn't run. The amount of thickening agent, if present, ranges from about 0.001-5%, preferably about 0.005-4%, more preferably about 0.005-3% by weight of the total composition.

A variety of thickening agents are suitable, including those set forth above with respect to the developer composition and in the same general ranges.

5. Solvents

It may be desirable to include one or more solvents in the dye composition. Such solvents assist in solubilizing the primary intermediate dyestuff and coupler dyestuff components, in addition to the other ingredients in the composition. The solvent is preferably present at about 0.01-10%, preferably 0.05-8%, more preferably 0.1-7% by weight of the total composition. Suitable solvents include those referred to as humectants with respect to the developer composition and in the same general ranges, as well as alkoxydiglycols such as ethoxydiglycol. The preferred solvent comprises ethoxydiglycol.

6. Chelating Agents

Preferably, the oxidative dye composition contains one or more chelating agents that are capable of chelating the metal ions found in water. If water contains too many extraneous metal ions they can interfere with the coloration process. Preferred ranges of chelating agent are 0.001-5%, preferably 0.005-4%, more preferably 0.01-3% by weight of the total composition. Preferred chelating agents are ELDTA, HEDTA, and sodium or potassium salts thereof, as set forth with respect to the developer composition.

7. Antioxidants and Preservatives

The oxidative dye composition may also contain one or more antioxidants such as BHA, BHT, sodium sulfite, and so on. Suggested ranges are from about 0.001-5%, preferably 0.005-4%, more preferably about 0.01-3% by weight of the total oxidative dye composition. Preservatives may also be incorporated into the oxidative dye composition.

In the method of the invention, from about 1 to 3 parts of the developer composition is combined from about 1 to 3 parts of the dye composition. The mixture is immediately applied to hair for the time necessary to color the hair. This time ranges from about 5 to 60 minutes, preferably from 10 to 45 minutes. The mixture is then rinsed off with water. In the most preferred embodiment, about 1.5 parts of the developer composition is combined with about 1 part of the oxidative dye composition. The mixture is applied to the hair for about 25-45 minutes, then rinsed out well with water. If desired, the hair may be treated with shampoo, conditioner, or both.

The invention will be further described in connection with the following examples which are set forth for purposes of illustration only.

### EXAMPLE 1

Aqueous oxidizing agent, or developer, compositions having hydrogen peroxide concentrations ranging from 10 to 30 were prepared as follows:

| | A | B | C | D |
|---|---|---|---|---|
| Ingredient | 10 Vol. | 15 Vol. | 20 Vol. | 30 Vol. |
| Water | QS | QS | QS | QS |
| Phosphoric acid | 0.08 | 0.08 | 0.02 | 0.02 |
| Disodium phosphate | 0.05 | 0.05 | 0.03 | 0.03 |
| Disodium EDTA | 0.02 | 0.02 | 0.02 | 0.02 |
| Methyl paraben | — | — | 0.05 | 0.05 |
| PEG-6 caprylic/capric glycerides | 18.00 | 18.00 | — | — |
| Cetearyl alcohol, Ceteareth-20 | — | — | 3.60 | 2.94 |
| Ceteareth-20 | — | — | 0.40 | 0.42 |
| Lauryl pyrrolidone | — | — | 2.00 | — |
| Sodium lauryl sulfate | — | — | — | 0.50 |
| Cyclomethicone, | — | — | 0.01 | 0.01 |
| trimelhylsiloxysilicate | | | | |
| Trimethylsilylamodimethicone, | — | — | 1.75 | — |
| Octoxynol-40, Isolaureth-6, | | | | |
| propylene glycol, water | | | | |
| Trimethylsilylamodimethicone, | — | — | — | 1.75 |
| C11-15 pareth-7, C12-16 pareth- | | | | |
| 9, trideceth-12, glycerin, water | | | | |
| Mineral oil | 2.50 | 2.50 | 0.60 | — |
| Coconut oil | 2.50 | 2.50 | — | — |
| Laureth-4 | 5.00 | 5.00 | — | — |
| Oleyl alcohol | 4.00 | 4.00 | — | — |
| Propylene glycol | 8.00 | 8.00 | — | — |
| Hydrogen peroxide (35% aqueous | 9.00 | 13.50 | 18.00 | 26.00 |
| solution) | | | | |
| Steareth-10 allyl ether/acrylates | — | — | 1.00 | 1.80 |
| copolymer | | | | |

Formulas A and B are in the microemulsion form, manufactured according to U.S. Pat. No. 6,315,989, incorporated by reference in its entirety. All the formulas were prepared by combining the ingredients and mixing well.

### EXAMPLE 2

Oxidative hair color compositions were prepared as follows

In all the four formulas below, QS the dyes and not disclose the exact amount as these are Colorsilk formulas along with the invention formulas and are proprietary to Revlon. I think best would be if you just mention and compare with the Colorsilk shade, that is, Medium brown and Extra light natural blonde and mention only the MLI listing of the ingredients, instead of the actual formula of Colosilk.:

| | | | C | D |
|---|---|---|---|---|
| | A | B | Extra | Extra |
| | Medium | Medium | Light | Light |
| | Brown | Brown | Blonde | Blonde |
| Ingredient | Mild | Regular | Mild | Regular |
| Water | QS | QS | QS | QS |
| Sodium sulfite | 0.50 | 1.00 | 0.50 | 1.00 |
| Erythobic acid | 0.20 | 0.20 | 0.20 | 0.20 |
| Oleic acid | 13.00 | 13.00 | 13.00 | 13.00 |
| Isopropyl alcohol | — | 7.00 | — | 8.50 |
| Ethoxydiglycol | 2.00 | | 2.00 | — |
| Potassium cocoyl hydrolyzed | 3.00 | 4.50 | 3.00 | 4.50 |
| collagen | | | | |
| Lauramide MEA | 5.50 | 3.00 | 5.50 | 3.00 |
| Sodium laureth sulfate (28% | 2.00 | 6.70 | 2.00 | 6.70 |
| aqueous solution) | | | | |
| PEG-2 cocamine | 6.50 | 4.50 | 6.50 | 4.50 |
| Laneth-5 | — | 0.80 | — | 0.80 |
| Lauryl pyrrolidone | — | — | — | 2.00 |
| Tetrasodium EDTA | 0.50 | 0.30 | 0.50 | 0.30 |
| Meadowfoam seed oil | — | 0.01 | — | 0.01 |
| Oleyl alcohol | — | 0.01 | — | 0.01 |
| Cetearyl alcohol | 3.00 | — | 3.00 | — |
| Steareth-21 | 1.00 | — | 1.00 | — |
| Ethanolamine | 8.00 | 8.00 | 13.50 | 10.75 |
| P-phenylenediamine | 0.59 | 0.48 | 0.02 | 0.03 |
| P-phenylenediamine sulfate | — | 0.01 | — | |
| Resorcinol | 0.51 | 0.21 | 0.02 | 0.03 |
| 4-chlororesorcinol | — | 0.02 | — | |
| P-aminophenol | 0.05 | 0.04 | 0.01 | 0.01 |
| 4-amino-2-hydroxytoluene | 0.04 | 0.02 | — | |
| M-aminophenol | 0.10 | 0.02 | — | 0.01 |
| Hypnea musciformis extract, | — | 0.01 | — | 0.01 |
| gellidiela acerosa extract, | | | | |
| sargassum filipendula extract, | | | | |
| sorbiol | | | | |
| Sodium benzotriazolyl | — | 0.01 | — | 0.01 |
| butylphenol sulfonate, buteth-3, | | | | |
| tributyl citrate | | | | |
| Wheat amino acids | 1.00 | 1.00 | 1.00 | 1.00 |
| Fragrance | 1.00 | 1.00 | 1.00 | 1.00 |

The compositions were prepared by combining the ingredients and mixing well. The compositions were stored in brown glass bottles or tubes.

### EXAMPLE 3

Oxidative hair color was applied to split heads on two salon panelists. Panelist 1 had substantially gray hair, having about 100% gray in the front, 40% gray on the crown, and 20% gray on the nape. Panelist 2 had blonde hair with about 10% gray at the front, and 5% gray at the crown and nape.

One side of each panelists head was treated with a mixture of one part of the oxidative hair color composition of Formula C from Example 2 and 1.5 parts of developer composition A from Example 1. The mixture was applied to half heads on both panelists for thirty minutes, then rinsed out well with water. The other half heads were treated with a mixture of 1 part of the oxidative dye of Formula D from Example 2 and 1.5 parts of developer C from Example 1 for 30 minutes.

A trained salon evaluator evaluated the resulting hair color on both panelists. In Panelist 1 the half head treated with the hair color process of the invention had slightly less lift, but the color was vibrant and true and the hair was well conditioned. The half head treated with the regular oxidative process exhibited better lift but hair was not as conditioned.

For Panelist 2 the half head treated with the hair color process of the invention exhibited better hair conditioning and good lift. The hair on the other half head was dryer and more raspy, but with true color.

### EXAMPLE 4

Salon panelists having naturally medium brown hair were split head tested. The panelists had varying degrees of gray hair. A hair color mixture obtained by combining 1 part of Formula A oxidative dye from Example 2 with 1.5 parts of the developer composition A from Example 2, was applied to the half heads for 30 minutes, then rinsed out well with water. The other half heads were treated with a mixture prepared by combining 1 part of the oxidative dye composition B from Example 2 and 1.5 parts of the developer composition C from Example 1. The mixture was applied to the hair for 30 minutes, then rinsed out well with water.

A trained salon evaluator evaluated the heads and noted that the half heads treated according to the method of the invention exhibited substantially improved conditioning. In general the color deposit and vibrancy on both sides was equivalent.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A water and oil emulsion developer composition for use in bleaching or oxidatively coloring hair comprising hydrogen peroxide in at least about 2-8% by weight of the total oil phase and from about 1 to 5% by weight of the total composition, wherein the ratio of the water to oil phase ranges from about 1.5 to 5 parts water phase to 1 part oil phase and the composition comprises greater than about 75% by weight of the total composition of water phase and less than about 25% by weight of the total composition of oil phase.

2. The composition of claim 1 wherein the oil phase comprises from about 15 to 80% by weight of the total composition of oil.

3. The composition of claim 1 wherein the water phase comprises from about 1 to 75% by weight of the total composition.

4. The composition of claim 1 wherein the oil phase has a Hildebrand solubility parameter ranging from about 7 to 9.

5. A method for oxidatively coloring hair comprising applying to the hair an oxidative hair color mixture prepared by combining at least one oxidative dye composition and at least one water and oil emulsion developer containing hydrogen peroxide from about 2 to 8% by weight of the total oil phase and 1 to 5% by weight of the total composition, and wherein the ratio of water to oil phase ranges from about 1.5 to 5 parts water phase to 1 part oil phase.

6. The method of claim 5 wherein the developer comprises from about 15 to 80% by weight of the total composition of oil.

7. The method of claim 6 wherein the oil comprises a hydrophobic oil.

8. The method of claim 7 wherein the oil comprises mineral oil, coconut oil, or mixtures thereof.

9. The method of claim 8 wherein the developer composition comprises from about 20-70% by weight of the total composition of oil

10. The method of claim 5 wherein the developer composition comprises from about 28 to 65% by weight of the total composition of oil.

11. The method of claim 5 wherein the developer is in the form or a water and oil microemulsion.

12. The method of claim 5 wherein the developer is in the form of a liquid crystalline emulsion composition.

13. The method of claim 5 wherein the oil phase ingredients together have a Hildebrand solubility parameter ranging from about 6.5 to 10.

14. The method of claim 5 wherein the oil phase ingredients together have a solubility parameter ranging from about 7 to 9.

15. The method of claim 5 wherein the water phase of the emulsion ranges from about 1 to 75% by weight of the total composition.

16. The method of claim 5 wherein the water phase of the emulsion ranges from about 5 to 70% by weight of the total composition.

17. The method of claim 5 wherein the ratio of the water phase to the oil phase in the developer ranges from about 1.5 to 5.0 to 1.

18. The method of claim 15 wherein the ratio of the water phase to the oil phase in the developer is from about 2 to 3 to 1.

19. The method of claim 16 wherein the ratio of the water phase to the oil phase in the developer is about 2.1.

20. The method of claim 5 wherein the oil phase comprises a hydrophobic oil.

21. The method of claim 5 wherein the oil comprises mineral oil, coconut oil, or mixtures thereof.

22. The method of claim 5 wherein the hair is colored in about 10 to 45 minutes.
